(19) United States
(12) Patent Application Publication
(10) Pub. No.: US 2026/0165629 A1
(43) Pub. Date: Jun. 18, 2026

(54) METHOD AND APPARATUS TO GENERATE IMAGERY REGARDING A PATIENT'S HEART

(71) Applicant: Varian Medical Systems, Inc., Palo Alto, CA (US)

(72) Inventors: Felix Meister, Erlangen (DE); Tiziano Passerini, Plainsboro, NJ (US); Puneet Sharma, Princeton Junction, NJ (US)

(21) Appl. No.: 18/978,177

(22) Filed: Dec. 12, 2024

(51) Int. Cl. A61B 5/339; A61B 5/367; A61B 90/00; G06T 15/04; G06T 17/20; G06T 19/20; G16H 20/40; G16H 50/50

(52) U.S. Cl. CPC A61B 5/339; A61B 5/367; G06T 15/04; G06T 17/205; G06T 19/20; G16H 20/40; G16H 50/50; A61B 2090/367; G06T 2210/41; G06T 2219/2021

(57) ABSTRACT

A control circuit accesses electroanatomic mapping (EAM) two-dimensional image information for a particular patient's heart and then generates a three-dimensional EAM presentation of the particular patient's heart as a function of the EAM two-dimensional image information to provide a generated three-dimensional EAM presentation of the particular patient's heart. The control circuit can then generate a treatment plan to treat a condition of the particular patient's heart using the generated three-dimensional EAM presentation of the particular patient's heart, and then administer treatment to the particular patient's heart using the treatment plan.

### TECHNICAL FIELD

These teachings relate generally to treating a patient's cardiac condition and more particularly to catheter-based ablation or cardiac radioablation.

### BACKGROUND

Ventricular tachycardia is a cardiac condition that can lead to sudden cardiac death if left untreated. Catheter-based ablation and/or cardiac radioablation are sometimes used in the management of ventricular tachycardias. Both of these treatments can serve to locally alter cardiac tissue to eliminate wave re-entry. Unfortunately, some of these procedures can result in complications and long-term success is only modest. The applicant has determined that accurate identification of the responsible pathways (for instance, by localizing the exit site of the ventricular tachycardias circuit) can be an important factor leading to a successful treatment.

The applicant has determined that electroanatomic mapping can serve a useful role in assessing the functional, electrophysiological behavior of cardiac tissue. Unfortunately, electroanatomic mapping data may not be available at the time of need, or, if available, may be stored in an incompatible format.

### BRIEF DESCRIPTION OF DRAWINGS

The above needs are at least partially met through provision of the method and apparatus to generate imagery regarding a patient's heart described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings;

FIG. 3 comprises a flow diagram as configured in accordance with various embodiments of these teachings;

FIG. 4 comprises a workflow diagram as configured in accordance with various embodiments of these teachings; and

FIG. 5 comprises a workflow diagram as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, pursuant to these various embodiments, a control circuit can access electroanatomic mapping (EAM) two-dimensional image information for a particular patient's heart and then generate a three-dimensional EAM presentation of the particular patient's heart as a function of the EAM two-dimensional image information to provide a generated three-dimensional EAM presentation of the particular patient's heart. The control circuit can then generate a treatment plan to treat a condition of the particular patient's heart using the generated three-dimensional EAM presentation of the particular patient's heart, and then administer treatment to the particular patient's heart using the treatment plan.

By one approach, the electroanatomic mapping two-dimensional image information can comprise two-dimensional screenshots (such as, for example, at least two different two-dimensional screenshots).

By one approach, the teachings will accommodate inputting information corresponding to the EAM two-dimensional image information into a neural network (such as, but not limited to, a convolutional neural network) that outputs a texture map and an initial three-dimensional mesh with texture coordinates that both correspond to the particular patient's heart. By one approach, that trained neural network can be trained with a training corpus comprising a plurality of synthetic EAM images that were generated using a variety of different anatomical and electrophysiological properties.

By one approach, these teachings will accommodate generating an approximate three-dimensional EAM model for the particular patient's heart as a function of the aforementioned texture map and the initial three-dimensional mesh, and then processing the texture map and the initial three-dimensional mesh via a differentiable renderer and an optimization routine to provide a more finely-tuned texture map and three-dimensional mesh that more closely match information comprising the EAM two-dimensional image information.

By one approach, these teachings will accommodate deforming the aforementioned approximate three-dimensional EAM model to three-dimensional cardiac geometry information to provide a deformed three-dimensional EAM model and then mapping the more finely-tuned texture map from the deformed three-dimensional EAM model to the three-dimensional cardiac geometry to provide the aforementioned generated three-dimensional EAM presentation of the particular patient's heart.

By one approach, these teachings can comprise a non-transitory computer-readable medium that stores a computer program that itself comprises instructions that, when the computer program is executed by a computer, causes the computer to carry out any or all of the steps, functions, and/or activities described herein, including but not limited to accessing electroanatomic mapping (EAM) two-dimensional image information for a particular patient's heart, generating a three-dimensional EAM presentation of the particular patient's heart as a function of the EAM two-dimensional image information to provide a generated three-dimensional EAM presentation of the particular patient's heart, generating a treatment plan to treat a condition of the particular patient's heart using the generated three-dimensional EAM presentation of the particular patient's heart, and then administering treatment to the particular patient's heart using the treatment plan.

So configured, these teachings can provide for reconstructing a three-dimensional EAM model from two-dimensional screen capture images. These teachings can be especially beneficial in use cases when digitized three-dimensional EAM data may not be directly available for loading (for example, when such data is unavailable or is stored in an incompatible format).

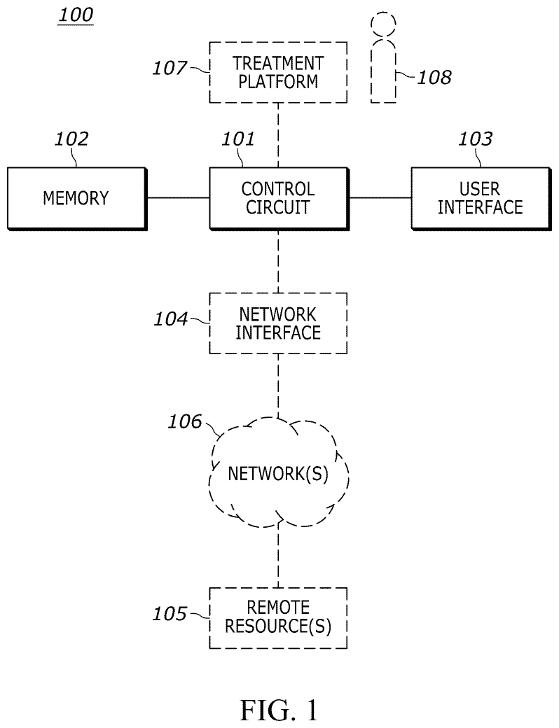

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

It will be appreciated that the control circuit 101 may comprise a single integrated platform or may comprise a plurality of such circuits that work in cooperation with one another.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101). As with the control circuit 101, the memory 102 may comprise a singular structure or may comprise a plurality of memory platforms that collectively comprise the “memory” of this apparatus 100.

In addition to information such as optimization information for a particular patient, information regarding a particular radiation treatment platform, and/or screen capture images as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

The control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also optionally couple to a network interface 104. So configured the control circuit 101 can communicate one or more remote resources 105 via one or more intervening networks 106.

By one approach, the control circuit 101 can operably couple to a treatment platform 107. This treatment platform 107 can be configured, for example, to treat ventricular tachycardia via ablation (using, for example, catheter-based ablation or cardiac radioablation). Ablation involves delivering radiofrequency energy, cryoenergy, or other forms of energy to targeted tissue in a patient's 108 heart to create small scars or lesions. This process can block abnormal electrical pathways, effectively curing the arrhythmia or at least significantly reducing its occurrence.

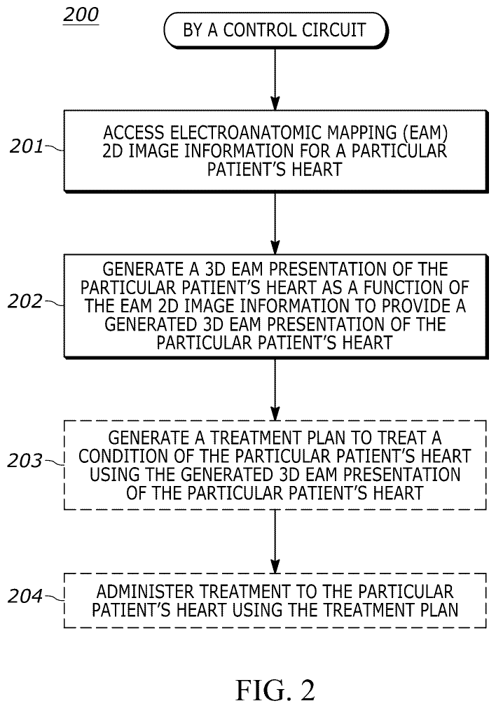

Referring now to FIG. 2, a process 200 that can be carried out, for example, in conjunction with the above-described application setting (and more particularly via the aforementioned control circuit 101) will be described.

At block 201, this process 200 provides for the control circuit 101 to access EAM two-dimensional image information for a particular patient's heart. The latter may comprise, for example, accessing the aforementioned memory 102. The accessed information may comprise two-dimensional screenshots and, at least for many application settings, will comprise two or more different two-dimensional screenshots. Multiple different images can be helpful, as a single two-dimensional image may not have sufficient information to recover the entire three-dimensional geometry of the patient's heart.

At block 202, the control circuit then generates a three-dimensional EAM presentation of the particular patient's heart as a function of the aforementioned EAM two-dimensional image information to thereby provide a generated three-dimensional EAM presentation of the particular patient's heart. (Examples in these regards are provided further herein.)

At optional block 203, the control circuit 101 may then generate a treatment plan to treat a condition of the particular patient's heart using the generated three-dimensional EAM presentation of the particular patient's heart. Various approaches are known in the art to generate such a treatment plan. As the present teachings are not overly sensitive to any particular selections in these regards, no further elaboration is provided here in these regards for the sake of brevity.

At optional block 204, the control circuit 101 can then serve to facilitate administering treatment to the particular patient's heart using the aforementioned treatment plan. Again, no further details in these regards need be provided here.

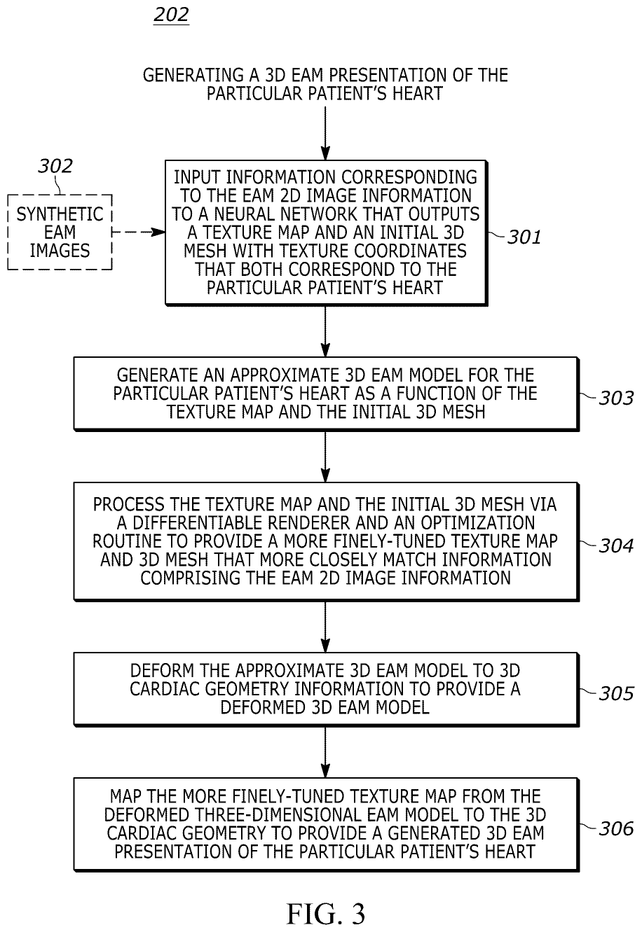

FIG. 3 provides some illustrative examples as regards generating the aforementioned three-dimensional EAM presentation of the particular patient's heart.

At block 301, the control circuit 101 inputs information corresponding to the EAM two-dimensional image information to a neural network that outputs a texture map and an initial three-dimensional mesh with texture coordinates that both correspond to the particular patient's heart. The neural network may comprise, for example, a convolutional neural network and may be trained with a training corpus comprising a plurality of synthetic EAM images 302 that were generated using a variety of different anatomical and electrophysiological properties.

At block 303, the control circuit 101 then generates an approximate three-dimensional EAM model for the particular patient's heart as a function of the texture map and the initial three-dimensional mesh. At block 304, the control circuit 101 processes the aforementioned texture map and the aforementioned initial three-dimensional mesh via a differentiable renderer and an optimization routine to provide a more finely-tuned texture map and three-dimensional mesh that more closely match information comprising the EAM two-dimensional image information.

At block 305, the control circuit 101 deforms the aforementioned approximate three-dimensional EAM model to three-dimensional cardiac geometry information to provide a deformed three-dimensional EAM model. That three-dimensional cardiac geometry information may comprise, for example, a mean shape model or patient-specific geometry that is derived from three-dimensional medical images for the patient (such as computed tomography images of the patient). At block 306, the control circuit 101 can then map the aforementioned more finely-tuned texture map from the aforementioned deformed three-dimensional EAM model to the aforementioned three-dimensional cardiac geometry to provide the generated three-dimensional EAM presentation of the particular patient's heart as referred to in block 202 of FIG. 2 above.

Further details that comport with these teachings will now be presented. It will be understood that the specific details of these examples are intended to serve an illustrative purpose and are not intended to suggest any particular limitations with respect to these teachings.

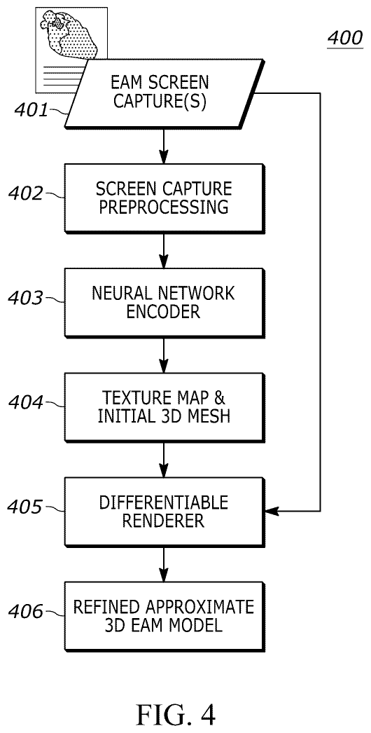

FIG. 4 presents an illustrative screen capture workflow 400. At block 401 a plurality of screen captures of EAM images of the patient's heart are accessed. By one approach, at least some, and preferably all, of these images each present the electroanatomic map in a known cardiac orientation.

The accessed images are then preprocessed at block 402. That preprocessing may comprise, for example, using a localizer to detect the EAMs in the image(s) to facilitate extracting the region(s) of interest. The preprocessing can also include using a segmentation algorithm to remove background content.

The preprocessed EAM screen captures are then input into a convolutional neural network 403 that has been trained to estimate and provide as output a texture map and an initial three-dimensional mesh with texture coordinates 404. In particular, a texture map and an initial, approximate three-dimensional EAM model can be inferred from the two-dimensional screen capture images. By one approach, this step may be realized by the aforementioned convolutional neural network acting as an encoder that produces a latent embedding that describes the object. A neural decoder then maps the latent embedding to a three-dimensional point cloud with each point having an assigned texture value. The latter are then converted into a mesh and a texture map using, for example, a meshing and rasterization method, respectively. By another approach, the decoding can be realized by two networks. A graph-neural-network can iteratively deform a template mesh and then a convolutional neural network can generate a three-dimensional texture map.

At block 405 a differentiable renderer is setup and an optimization routine is applied to fine-tune the texture map and the three-dimensional mesh to match the screen-captured EAM imagery. A differentiable renderer is a type of rendering algorithm used in image processing that enables the calculation of gradients of rendering outcomes with respect to their input parameters. By being differentiable, the renderer supports the backpropagation of errors, making it possible to adjust the input parameters (such as object orientations or geometry of the scene) in a way that the rendered image better matches a target image or optimizes some defined loss function. At block 405, the differentiability of the entire pipeline can be used together with an image-similarity loss to minimize differences between the synthetically rendered image and the EAM screen captures by deforming the mesh and fine-tuning the texture map with an appropriate optimization algorithm (such as gradient-descent-based optimization). The optimization can be stopped once the images match up to a certain threshold based on a similarity metric. The differentiable renderer 405 ultimately outputs a refined approximate three-dimensional EAM model 406.

While the resulting refined approximate three-dimensional EAM model 406 may already be used for comprehensive three-dimensional analysis, mapping the data to a three-dimensional cardiac mesh may further facilitate visual comprehension (at least in part because the acquired electroanatomic maps may include distortion and/or under-sampling artifacts). Accordingly, by one approach, these teachings may provide for additionally providing a mapping module that combines a differentiable renderer and a deformation neural network.

By one approach, a mean shape or patient-specific three-dimensional cardiac model can be used in conjunction with that differentiable renderer, pre-initialized with known acquisition orientations as input, for example, by the user. Within another optimization routine a deformation network can be coupled to a differentiable renderer to optimize the camera orientation and to deform the refined approximate three-dimensional EAM model 406 to the three-dimensional cardiac mesh. Once converged, the EAM texture can be mapped to the three-dimensional cardiac mesh. Given the inferred EAM model and corresponding 3D cardiac geometry (comprising, for example, a mean shape model or patient-specific geometry derived from 3D medical images), the deformation network can estimate an ordinary differentiable equation for the deformation of each vertex of the EAM model that brings the geometry closer to each other. Realizing the deformation as a neural differentiable equation enables a bijective deformation that guarantees that two vertex deformation trajectories cannot cross, thus, preserving the topological structure of the EAM model.

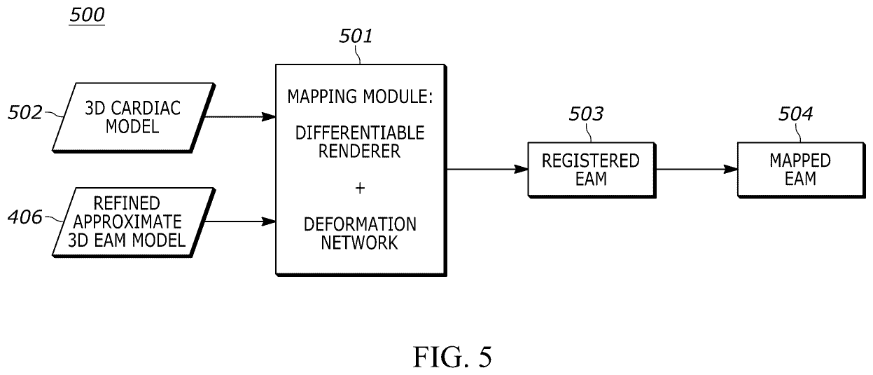

FIG. 5 presents an illustrative workflow 500 in the foregoing regards.

A mapping module 501 as described above receives as input the aforementioned refined approximate three-dimensional EAM model 406 as well as a three-dimensional cardiac model 502 for the patient. The differentiable renderer can be setup to match the known camera orientation of the EAM views and thereby enable querying the visible vertices of the EAM model. The latter allows restricting the deformation to only these vertices and helps with avoiding deformation artifacts.

The mapping module 501 can be iteratively applied until the EAM model is at least close to the three-dimensional cardiac model (for example, within some predetermined distance threshold) to yield a registered three-dimensional EAM 503. The texture data of the EAM model can then be mapped to the closest vertices in the three-dimensional cardiac model and geodesic interpolation can be applied to interpolate the values for intermediate vertices to yield a mapped three-dimensional EAM 504.

Training such a system can be a two-step process. The first step comprises preprocessing screen captured EAMs and inferring a three-dimensional EAM model estimate. Then, the three-dimensional EAM estimates can be mapped to the three-dimensional cardiac model. Since it is generally challenging to acquire large amounts of high-quality clinical data, cardiac computational modeling can be applied to generate a large training database of realistic and physiologically plausible EAMs. In particular, a large variety of EAMs can be simulated by varying anatomical and electrophysiological properties. Moreover, non-uniform randomized sampling of the endocardial surface and pre-computed cardiac contraction cycles can be employed to augment the set of EAMs. The computational model can provide the three-dimensional EAM, and multiple two-dimensional screen shots can be generated by placing a virtual camera in various positions and with various orientations.

So configured, these teachings can provide for the automatic projection of EAM information that is present in two-dimensional screen captures onto three-dimensional cardiac models by reconstructing a three-dimensional model with associated data. Since only screen captures of the EAM measurements are required, these teachings can leverage EAM information generated by essentially any system or manufacturer.

Further aspects of these teachings are provided by the subject matter of the following clauses (where it will be understood that any of these clauses can be combined with any one of more of the other clauses as appropriate).
Clause 1. A method comprising: by a control circuit: accessing electroanatomic mapping (EAM) two-dimensional image information for a particular patient's heart; generating a three-dimensional EAM presentation of the particular patient's heart as a function of the EAM two-dimensional image information to provide a generated three-dimensional EAM presentation of the particular patient's heart.
Clause 2. The method of clause 1 wherein accessing EAM two-dimensional image information for the particular patient's heart comprises accessing two-dimensional screen shots.
Clause 3. The method of either of clause 1 or 2 wherein accessing two-dimensional screen shots comprises accessing at least two different two-dimensional screen shots.
Clause 4. The method of any of clauses 1-3 further comprising: inputting information corresponding to the EAM two-dimensional image information to a neural network that outputs a texture map and an initial three-dimensional mesh with texture coordinates that both correspond to the particular patient's heart.
Clause 5. The method of clause 4 wherein the neural network comprises a trained neural network that has been trained with a training corpus comprising a plurality of synthetic EAM images that were generated using a variety of different anatomical and electrophysiological properties.
Clause 6. The method of clause 5 wherein the neural network comprises a convolutional neural network.
Clause 7. The method of clause 4 further comprising: generating an approximate three-dimensional EAM model for the particular patient's heart as a function of the texture map and the initial three-dimensional mesh.
Clause 8. The method of clause 7 further comprising: processing the texture map and the initial three-dimensional mesh via a differentiable renderer and an optimization routine to provide a more finely-tuned texture map and three-dimensional mesh that more closely match information comprising the EAM two-dimensional image information.
Clause 9. The method of clause 8 further comprising: deforming the approximate three-dimensional EAM model to three-dimensional cardiac geometry information to provide a deformed three-dimensional EAM model; mapping the more finely-tuned texture map from the deformed three-dimensional EAM model to the three-dimensional cardiac geometry to provide the generated three-dimensional EAM presentation of the particular patient's heart.
Clause 10. The method of any of clauses 1-9 further comprising: generating a treatment plan to treat a condition of the particular patient's heart using the generated three-dimensional EAM presentation of the particular patient's heart; administering treatment to the particular patient's heart using the treatment plan.
Clause 11. An apparatus comprising: a control circuit configured to: access electroanatomic mapping (EAM) two-dimensional image information for a particular patient's heart; generate a three-dimensional EAM presentation of the particular patient's heart as a function of the EAM two-dimensional image information to provide a generated three-dimensional EAM presentation of the particular patient's heart.
Clause 12. The apparatus of clause 11 wherein the control circuit is configured to access EAM two-dimensional image information for the particular patient's heart by accessing two-dimensional screen shots.
Clause 13. The apparatus of either of clause 11 or 12 wherein the control circuit is configured to access two-dimensional screen shots by accessing at least two different two-dimensional screen shots.
Clause 14. The apparatus of any of clauses 11-13 wherein the control circuit is further configured to: input information corresponding to the EAM two-dimensional image information to a neural network that outputs a texture map and an initial three-dimensional mesh with texture coordinates that both correspond to the particular patient's heart.
Clause 15. The apparatus of clause 14 wherein the neural network comprises a trained neural network that has been trained with a training corpus comprising a plurality of synthetic EAM images that were generated using a variety of different anatomical and electrophysiological properties.
Clause 16. The apparatus of clause 15 wherein the neural network comprises a convolutional neural network.
Clause 17. The apparatus of clause 14 wherein the control circuit is further configured to: generate an approximate three-dimensional EAM model for the particular patient's heart as a function of the texture map and the initial three-dimensional mesh.
Clause 18. The apparatus of clause 17 wherein the control circuit is further configured to: process the texture map and the initial three-dimensional mesh via a differentiable renderer and an optimization routine to provide a more finely-tuned texture map and three-dimensional mesh that more closely match information comprising the EAM two-dimensional image information.
Clause 19. The apparatus of clause 18 wherein the control circuit is further configured to: deform the approximate three-dimensional EAM model to three-dimensional cardiac geometry information to provide a deformed three-dimensional EAM model; map the more finely-tuned texture map from the deformed three-dimensional EAM model to the three-dimensional cardiac geometry to provide the generated three-dimensional EAM presentation of the particular patient's heart.
Clause 20. The apparatus of any of clauses 11-19 wherein the control circuit is further configured to: generate a treatment plan to treat a condition of the particular patient's heart using the generated three-dimensional EAM presentation of the particular patient's heart; administer treatment to the particular patient's heart using the treatment plan.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

1. A method comprising:
by a control circuit:
accessing electroanatomic mapping (EAM) two-dimensional image information for a particular patient's heart;
generating a three-dimensional EAM presentation of the particular patient's heart as a function of the EAM two-dimensional image information to provide a generated three-dimensional EAM presentation of the particular patient's heart.

2. The method of claim 1 wherein accessing EAM two-dimensional image information for the particular patient's heart comprises accessing two-dimensional screen shots.

3. The method of claim 2 wherein accessing two-dimensional screen shots comprises accessing at least two different two-dimensional screen shots.

4. The method of claim 1 further comprising:
inputting information corresponding to the EAM two-dimensional image information to a neural network that outputs a texture map and an initial three-dimensional mesh with texture coordinates that both correspond to the particular patient's heart.

5. The method of claim 4 wherein the neural network comprises a trained neural network that has been trained with a training corpus comprising a plurality of synthetic EAM images that were generated using a variety of different anatomical and electrophysiological properties.

6. The method of claim 5 wherein the neural network comprises a convolutional neural network.

7. The method of claim 4 further comprising:
generating an approximate three-dimensional EAM model for the particular patient's heart as a function of the texture map and the initial three-dimensional mesh.

8. The method of claim 7 further comprising:
processing the texture map and the initial three-dimensional mesh via a differentiable renderer and an optimization routine to provide a more finely-tuned texture map and three-dimensional mesh that more closely match information comprising the EAM two-dimensional image information.

9. The method of claim 8 further comprising:
deforming the approximate three-dimensional EAM model to three-dimensional cardiac geometry information to provide a deformed three-dimensional EAM model;
mapping the more finely-tuned texture map from the deformed three-dimensional EAM model to the three-dimensional cardiac geometry to provide the generated three-dimensional EAM presentation of the particular patient's heart.

10. The method of claim 1 further comprising:
generating a treatment plan to treat a condition of the particular patient's heart using the generated three-dimensional EAM presentation of the particular patient's heart;
administering treatment to the particular patient's heart using the treatment plan.

11. An apparatus comprising:
a control circuit configured to:
access electroanatomic mapping (EAM) two-dimensional image information for a particular patient's heart;
generate a three-dimensional EAM presentation of the particular patient's heart as a function of the EAM two-dimensional image information to provide a generated three-dimensional EAM presentation of the particular patient's heart.

12. The apparatus of claim 11 wherein the control circuit is configured to access EAM two-dimensional image information for the particular patient's heart by accessing two-dimensional screen shots.

13. The apparatus of claim 12 wherein the control circuit is configured to access two-dimensional screen shots by accessing at least two different two-dimensional screen shots.

14. The apparatus of claim 11 wherein the control circuit is further configured to:
input information corresponding to the EAM two-dimensional image information to a neural network that outputs a texture map and an initial three-dimensional mesh with texture coordinates that both correspond to the particular patient's heart.

15. The apparatus of claim 14 wherein the neural network comprises a trained neural network that has been trained with a training corpus comprising a plurality of synthetic EAM images that were generated using a variety of different anatomical and electrophysiological properties.

16. The apparatus of claim 15 wherein the neural network comprises a convolutional neural network.

17. The apparatus of claim 14 wherein the control circuit is further configured to:
generate an approximate three-dimensional EAM model for the particular patient's heart as a function of the texture map and the initial three-dimensional mesh.

18. The apparatus of claim 17 wherein the control circuit is further configured to:
process the texture map and the initial three-dimensional mesh via a differentiable renderer and an optimization routine to provide a more finely-tuned texture map and three-dimensional mesh that more closely match information comprising the EAM two-dimensional image information.

19. The apparatus of claim 18 wherein the control circuit is further configured to:
deform the approximate three-dimensional EAM model to three-dimensional cardiac geometry information to provide a deformed three-dimensional EAM model;
map the more finely-tuned texture map from the deformed three-dimensional EAM model to the three-dimensional cardiac geometry to provide the generated three-dimensional EAM presentation of the particular patient's heart.

20. The apparatus of claim 11 wherein the control circuit is further configured to:
generate a treatment plan to treat a condition of the particular patient's heart using the generated three-dimensional EAM presentation of the particular patient's heart;
administer treatment to the particular patient's heart using the treatment plan.
